(19) United States
(12) Patent Application Publication
(10) Pub. No.: US 2003/0219890 A1
(43) Pub. Date: Nov. 27, 2003

(54) Probe array bio-analysis by centrifuging shallow reaction cell

(76) Inventors: Gary B. Gordon, Saratoga, CA (US); Magdalena Anna Ostrowski, San Jose, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/152,949

(22) Filed: May 21, 2002

(51) Int. Cl. C12M 1/34

(52) U.S. Cl. 435/287.2; 435/287.9; 435/288.3; 435/293.1; 422/72; 435/6; 436/45

(57) ABSTRACT

A bioanalytic method replenishes depleted zones of a sample liquid in a shallow probe-array reaction cell under ultragravity centrifugal forces. The ultragravity overcomes viscous and surface-tension forces to permit replenishment despite a shallow reaction-cell depth of 25 microns. Thus, replenishment is achieved using 1/10^{th} the sample volume normally used in probe-array systems that use mixing to facilitate binding reactions. For similar amounts of sample, the shallow cell takes advantage of a ten-times greater concentration to achieve much greater signal strengths in much shorter times. Thus, signal strengths that normally take 17 hours to achieve are achieved in about 60 minutes.

### BACKGROUND OF THE INVENTION

The present invention relates to analysis of biological samples and, more particularly, to such analysis using an array of probes. A major objective of the invention is to provide for faster sample analysis using smaller volumes of sometimes-scarce tissue samples.

Many of the miracles of modern medical science can be traced to advances in the analysis of bio-molecules. In particular, advances in DNA analysis has allowed the human genome to be decoded and continues to offer the prospect of cures for a wide range of diseases. One class of bio-analytical techniques uses biological probes to selectively bind to respective target molecules in the sample. Various techniques, including fluorescent marking of target molecules, can be used to detect the presence of target molecules bound to a probe, and thus the presence of the corresponding component in the original sample. Furthermore, the concentration of the sample components in the original sample can be determined by measuring the number or quantity of target molecules that have bound to respective probes.

As effective as bio-analytic techniques have become, the long durations involved have burdened medical advances and have proved a liability in diagnosing individual patients. It is often necessary to test for thousands of sample components, but the prospect of such a large number of prolonged analyses can be daunting. Results can be analyzed before reactions are completed, but only at the cost of detection and measurement sensitivity, which is often critical. In addition, analyses often face limited amounts of sample, which is often derived from tissue, making it more difficult to perform large numbers of tests even where time and equipment are not limiting factors.

Probe arrays represent considerable progress in allowing large number of analyses to be performed concurrently on a small amount of sample. A probe array is typically a two-dimensional array of probes bound to a surface. Each probe can be used to quantify a different potent sample component, so that many distinct analyses can be performed concurrently on a single sample.

In a cover-slip approach, 25-100 microliters (μl) of liquid sample is placed on a glass slide, which is then covered with another glass slide bearing a probe array. The structure is held in place by the viscosity and surface tension associated with the liquid sample. In the cover slip approach, the sample liquid itself spaces the slides. However, this approach does not ensure the slide surfaces are parallel, so that non-uniformities can be introduced. An alternative is to use lift-slips, in which the cover has Teflon ridges around the probe array. The rigid Teflon ridges maintain the parallelism of the base and cover as it spaces them. The assembled reaction cell can be open at the sides, four corners, or a pair of diagonally opposing corners to permit bubbles to escape.

Once the sample is in contact with the array, each probe binds with corresponding target molecules in its vicinity. As target molecules are bound, the probe vicinity is depleted of the target molecule, slowing the reaction rate. The vicinity is replenished by diffusion from other regions of the sample liquid, but only at a rate of about 5 millimeters (mm) per day. Given typical array dimensions of 2 cm×2 cm, (“cm”=“centimeters”), it can take days before binding levels off at some maximum.

Mixing can maintain a uniform sample distribution, minimizing local depletion and thereby increasing reaction rates. However, mixing is problematic at small volumes due to surface tension effects. To facilitate mixing, a sample can be diluted, e.g., to 500 μl, to achieve a greater hydraulic diameter, thereby decreasing surface-tension effects. With such a volume, mixing can be achieved by mechanical manipulation, e.g., shaking, of the reaction cell. One commercial hybridizer, manufactured by Affymetrix (Santa Clara, Calif.) uses active pumping to move the target solution. Another approach is to use jets of air to agitate the sample. Rubber seals can be used between a base and a cover to maintain the sample in position adjacent the array during agitation. U.S. Pat. Nos. 6,361,486 and 6,309,875 to Gordon disclose the use of a centrifuge to further reduce the effects of surface tension and viscosity to promote turbulent and thus more thorough mixing.

While mixing increases the reaction rate, this gain is partially offset by a lower reaction rate associated with the more dilute sample. As an alternative, 500 μl of undiluted sample can be used. However, since the sample is often derived from tissue, this amount of sample is not always available. Even if available, it is generally desirable to use less sample for a given analysis.

Surface tension can be reduced by adding surfactant. This technique allows smaller sample volumes, e.g., 250 μl to be mixed. However, this volume is still larger than desirable in many cases. Adding surfactant does not always achieve a satisfactory reduction in surface tension. Also, the surfactant may not be appropriate for all samples and buffers that might be used in an analysis. Furthermore, surfactant can interfere with the probe interactions for some sample components, so it is not always feasible to reduce volume requirements using this approach.

Some mixing techniques have been introduced or proposed to enable mixing of smaller volumes. Electric fields can be used with the intrinsic charge on DNA to propel molecules and agitate the sample. Nanogen, Inc., (San Diego, Calif.) teaches that such an approach to agitation is effective with small sample volumes. However, the currents involved introduce electrochemical activity, producing undesirable electrolysis products such as acids. Another method induces ultrasonic waves in the array substrate; however, this approach has not proved commercially feasible.

The most successful of the mixing approaches have decreased reaction times to about 17 hours. Typically, reactions begun one day are completed by the next. Still, overnight latencies are clearly undesirable, especially while a patient is waiting for a diagnosis or a series of analyses requires the results of one analysis before proceeding to the next. Further improvements in reaction rates are needed for probe arrays without decreasing sensitivity.

### SUMMARY OF THE INVENTION

The present invention provides for inducing a replenishment motion in a sample contained in a shallow reaction cell subjected to ultragravity centrifugal forces. The reaction cell can have a ridge that spaces a base and a cover, and also provides a barrier to prevent sample from escaping the reaction cell while it is reacting during centrifuging. The ridge can define a closed figure (and thus a complete seal) or an open figure (thus providing an opening for introducing and evacuating sample and rinse fluids) that encloses a probe array. (An open figure encloses a probe array if the closed figure defined by the open figure plus a line segment connecting its ends encloses the array.) The average depth of the reaction cell at the probe array is less than 200 ρl and, preferably, less than 50 μm or at least less than 100 μl. The ridge can be of compliant material so that it can serve as a complete or partial seal while under compression between the base and cover.

A centrifuge is used to generate the centrifugal forces that exceed 10 g (1 g equals the force of Earth's gravity at its surface). The ultra-gravity centrifugal force helps overcome the resistance to sample motion associated with the sample's viscosity and surface tension. More specifically, the ultragravity is used to keep the sample squarely located over the array, free of bubbles, and free of “skipped” areas caused by surface tension and varying surface properties causing the sample to only selectively “wet” the surface. Thus, the ultragravity makes it practical to induce a replenishment motion in small volumes, e.g., 10-200 microliters. The replenishment motion can be induced by any one of a variety of alternatives, for example, by rocking a reaction cell during centrifuging.

The ridge can be formed on the base, or on the cover, or be a separate element. The base can have a well into which sample liquid is inserted prior to assembly of the reaction cell. Assembly of the reaction cell involves installing a cover plate, typically bearing the probe array. If the ridge is open, the opening can be used for inserting and/or removing liquid, e.g., sample liquid or rinse liquid. The assembled reaction cell can be centrifuged. Mixing can be induced during centrifuging using any of a variety of techniques, including rocking the reaction cell using a second rotational axis provided by the centrifuge. Alternatively, cell compression, ultrasound, electric fields, or pumping can be used to mix the sample liquid, in conjunction with the centrifugation.

In the course of the present invention, it was discovered that the potential reaction rate gains achieved by the various mixing approaches are largely offset by the dilution of the sample (to make the sample liquid “mixable” under normal gravity). While prior-art small-volume approaches are limited by the absence or the ineffectiveness of mixing, and, while the prior-art mixing approaches tend to be limited by low-concentrations or excessively large sample quantity requirements, the present invention provides for rapid attainment of strong signals using small sample quantities.

In addition to providing for stronger detection signals in shorter times, the present invention provides for more robust detection of weakly expressed sample components. While free target molecules bind to the probes, bound target molecules are released into the sample fluid. The release rate correlates with the number of target molecules bound to the probe and the local concentration of the target molecules in the sample liquid. When the rate at which target molecules are being bound to a probe equals the number of molecules being released from the probe, the probe is in an equilibrium that represents a maximum signal strength for that sample component. Relative to probe methods that use more dilute samples, the present invention provides for a stronger maximum signal. Relative to the prior-art small volume approaches, the replenishment motion of the invention allows maximum signal strength to be reached in hours instead of days.

The present invention does not strive for the thorough turbulent mixing disclosed in U.S. Pat. No. 6,309,875 to Gordon. Instead, laminar flow combined with vertical diffusion suffices for replenishment given the shallowness of the reaction cell. Thus, the invention combines the advantages of high concentration and replenishment to provide the improved reaction rates.

The present invention provides for higher reaction rates generally. The higher reaction rates can be used to achieve fixed signal strengths more quickly or to achieve stronger signals within a relatively short time. In addition, the invention provides greater maximum sensitivity than is provided by the prior-art mixing approaches. Furthermore, the improvements in sensitivity and speed are achieved using small sample volumes, taking advantage of even small-volume samples. Finally, the invention achieves these ends while keeping the sample in a contiguous bubble-free volume above the array. These and other features and advantages of the invention are apparent from the description below with reference to the following drawings.

### BRIEF DESCRIPTION OF THE DRAWINGS

FIG. 1 is a schematic illustration of a bioanalytic system in accordance with the present invention.

FIG. 2 is a flow chart of a method of the invention practiced using the system of FIG. 1.

FIG. 3 is a graph comparing the performance of the present invention against two alternative methods using large reaction cells.

FIG. 4 is a photograph of a sample distribution in the reaction cell of FIG. 1 in the absence of centrifugal force.

### DETAILED DESCRIPTION

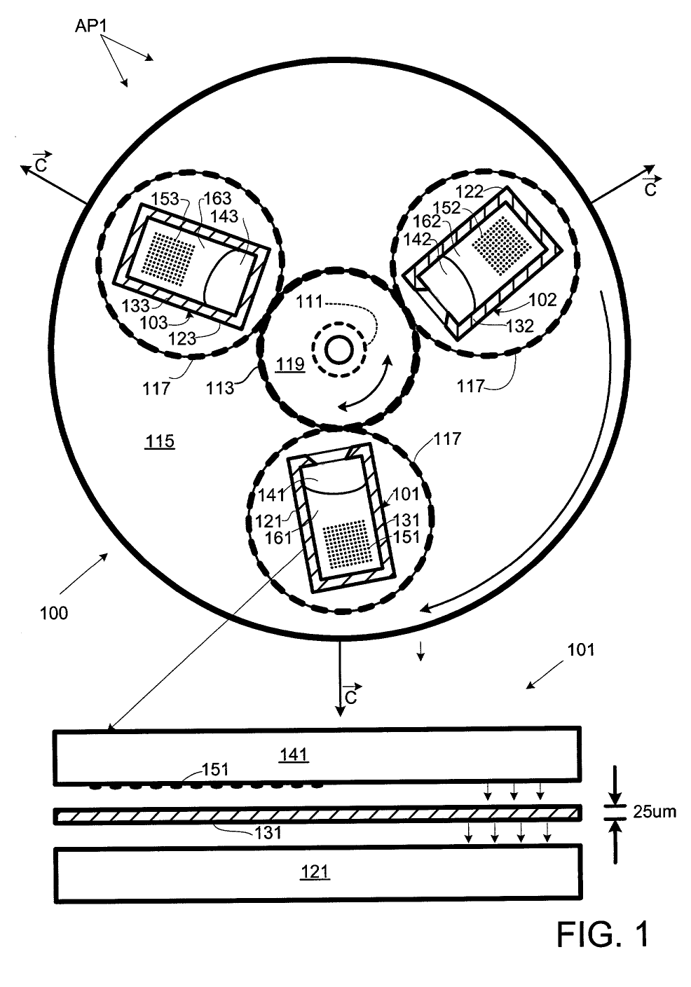

A bio-analytic system AP1 comprises a centrifuge 100, and three reaction cells 101, 102, and 103, as shown in FIG. 1. Centrifuge 100 uses a coaxial drive to provide both a centrifuge motion and a rocking motion. More specifically, centrifuge has an inner drive shaft 111 that extends along the axis of a hollow outer drive shaft 113. A respective servomotor drives each drive shaft so that their rotation rates can be controlled independently. Outer drive shaft 113 is coupled to a centrifuge rotor 115, on which reaction-cell mounting plates 117 are pivotably mounted. Inner drive shaft 111 is rigidly coupled to a gear 119, which is engaged with the mounting plates 117; accordingly, the orientation of each mounting plate 117 relative to a local centrifugal force vector C is determined by the orientation of inner drive shaft 111.

Each reaction cell 101, 102, 103, comprises a base 121, 122, 123, a gasket 131, 132, 133, and a cover plate 141, 142, 143. Each cover plate 141, 142, 143 bears a respective 2 cm×2 cm, 100×100-probe array 151, 152, 153. After assembly, each reaction cell 101, 102, 103, can hold a respective sample fluid 161, 162, 163.

Gaskets 131, 132, and 133 are formed by syringing silicone monomer directly on the respective base and then polymerizing to provide a compliant ridge. The gaskets determine the spacing of the respective base and cover plate for an assembled reaction cell. The present invention requires that the average spacing between the base and slide over the array be less than 200 microns. For reaction cells 101, 102, and 103, this average “depth” is 25 microns. This is an order of magnitude less than for most reaction cells that provide for mixing. The depth is comparable to that obtainable using the cover-slip and lift-slip approaches.

In addition to establishing the reaction-cell depths, gaskets 131, 132, and 133 are compliant so that, under compression between base and cover, they seal their respective reaction chambers (the interiors of the reactions cells) from liquid loss during centrifuging and mixing. Herein, a material is “compliant” if it is less than 80 a on the Shore “A” scale (http://www.calce.umd.edu/general/Facilities/Hardness_ad_.htm#3.5).

Gasket 133 has a closed form and forms a complete seal for reaction cell 103. Gaskets 131 and 132 have openings at one end to permit more convenient manual and automated filling and purging of reaction cells 101 and 102. During hybridization or other binding reaction, the opening is oriented generally “upward” in the local ultragravity field. However, the opening can be oriented downward to purge depleted sample liquid after the reaction is completed. Likewise, it can be oriented downward to purge wash fluid.

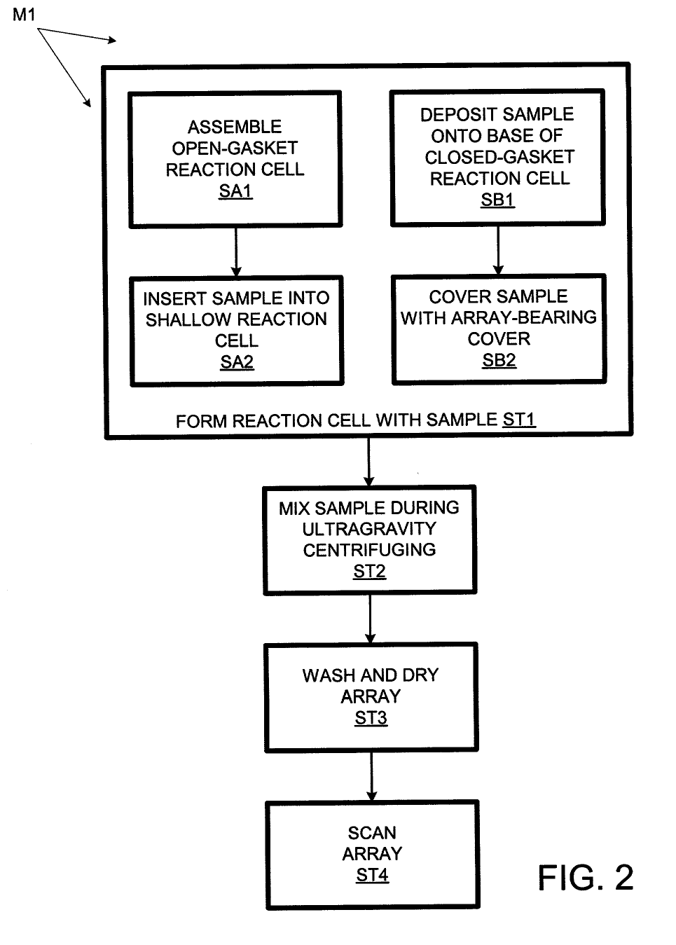

A method M1 of the invention using bio-analytic system AP1 is flow charted in FIG. 2. Step ST1 involves forming a reaction cell containing sample liquid. For a closed reaction cell such as cell 103, silicone monomer can be syringed onto the base to form a closed figure. After the silicone is cured to form a gasket, sample can be introduced onto the base, as indicated at substep SA1. Then the cover (with the array) can be seated on the gasket. This assembly can then be mounted on the centrifuge. Alternatively, some of the assembly can occur with the base on the centrifuge.

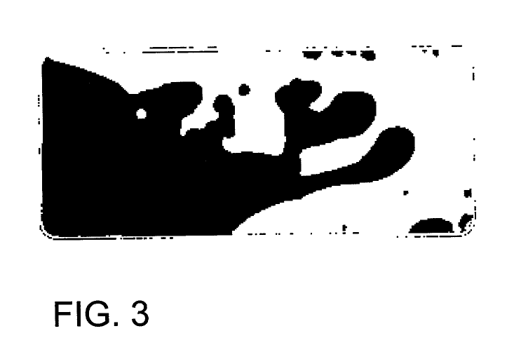

The distribution of the sample liquid prior to centrifuging is shown in FIG. 3. Note that the sample distribution is haphazard and discontiguous. Certain regions of the cell are not wet by the sample, and bubbles are enclosed within the sample liquid. Centrifuging removes the bubbles and forces the sample liquid into a contiguous volume located squarely over the array.

The foregoing steps can be used with an open reaction cell such as cell 101, substeps SA1 and SA2 can also be used. However, an open cell can be assembled, as at substep SB1 before sample liquid is introduced, e.g., by pipetting through the cell opening, as at substep SB2. The open reaction cell is suitable for automated sample injection.

The reaction cell and sample are then centrifuged so that the sample fluid is mixed under ultragravity (>10 g) conditions at step ST2. To this end, centrifuge 100 is spun at a rate sufficient to induce a 100 g force on the sample liquid. The mixing is performed by rocking the reaction cell by accelerating and decelerating inner drive shaft 111 relative to outer drive shaft 113 while the later drives centrifuge rotor 115. This mixing and centrifuging can continue for a “sufficient” reaction interval, e.g., 100 minutes. What is sufficient, of course, depends on the nature of the sample.

Centrifugation prevents a problem with conventional mixing, that bubbles form over areas either somewhat hydrophobic, or areas that just dry out. Once they form, they are difficult to dislodge, if the chamber is thin. The likelihood of a bubble dislodging increases with its buoyancy, but decreases with its area in the plane of the chamber. If a chamber is made half as thick, a bubble of a given diameter in the plane will have the same propensity to stick, because its area is the same; however it will only have half the buoyancy, the force tending to dislodge it. It is seen that with thinner chambers, the likelihood of a bubble sticking increases in inverse proportion to the thickness of a chamber. This is why the practical limit is of the order of 250-500 microns for mixing using only gravity. If the chamber is thinner, bubbles are likely to form and stick to the surface, disallowing mixing under them, or even access of the sample to the portion of the underlying array.

Once the reaction is completed, the array can be washed and dried at step ST3. This can be a simple process of removing the cover from the reaction cell, dipping it into a buffer and allowing the excess buffer to drip off the cover. However, an open cell can be partially inverted during centrifuging to force depleted sample liquid from the cell without disassembly. Furthermore, wash fluid can be introduced through the opening and a washing action can be achieved by using the inner drive shaft to rock the cell in its normal “upright” orientation. Then cell can be inverted to remove the used wash fluid. The wash cycle can be repeated as often as necessary. After the last wash is purged, centrifuging can continue to dry the array.

Once the array is properly washed and dried, it can be read in an array scanner. For example, the sample fluid can include fluorescent markers. Provided a sufficient number of fluorescent target molecules bind to a probe, the target can be detected. This detection (or lack of it) can be performed for all 10,000 probes in a single scan.

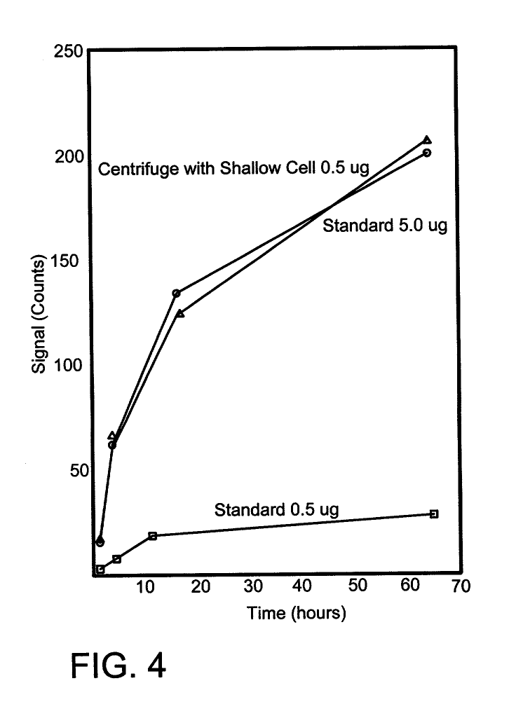

FIG. 4 is a graph showing the performance of the present invention using the shallow reaction cell versus two examples using large reaction cells. Signal strength is measured in counts, with 50 counts corresponding to about one dye molecule. The graph indicates that the invention equals the performance of a large-cell system using 1/10^{th} the sample volume. (Apparent differences between the high-concentration cases in the graph of FIG. 4 are considered insignificant.) When the invention uses the same amount of sample but at ten times the concentration, it provides greater signal strength in less time and greater final signal strength. These advantages can be exchanged so that the invention can provide greater sensitivity in less time using less sample.

The present invention provides for a variety of replenishment motions to be used with centrifugation. While the invention provides for complete mixing, laminar flow in conjunction with diffusion can suffice and is more readily achieved. The replenishment motion can be achieved by rocking (tilting back and forth) the reaction cell relative to the centrifugal force, i.e., the ultragravity field. Alternatively, the replenishment motion can be induced by flexing the sides of the reaction cell. This flexing can be achieved readily by increasing and decreasing the centrifuge rotation rate. A compliant base can be used to accommodate fluid moving slightly radially as the speed and centrifugal force are varied. Also, the compression can be achieved mechanically, without varying the centrifuge rate. The sample can be “poured” into and out of a well within the reaction cell by re-orienting the reaction cell in the ultragravity field. Finally, other mixing techniques such as using sound, ultrasound or electrophoresis to move the sample can be used in the context of centrifugation. Although thorough mixing is not required, any technique know to achieve thorough mixing can be applied in the present context as well.

While the invention has been described for a particular reaction cell geometry and specific technique for replenishment, it provides for a wide range of reaction cell geometries and replenishment techniques. The invention is applicable to a wide range of target molecules, including RNA, DNA, peptides, and proteins. In addition, a range of centrifuge rates can be used to overcome the viscosity and surface tension forms that would otherwise prevent mixing of the low volume of sample fluid. The replenishment can involve pivoting or shaking the chamber, during ultrasound or oscillating electric fields, or fluid pumps or air jets. These and other variations upon and modifications to the disclosed embodiments are provided by the present invention, the scope of which is defined by the following claims.

1. A bio-analytic reaction system comprising:
an array of bioactive probes; and
a reaction cell having a probe surface, an opposing surface, and
a ridge, said probe surface having a probe region on which said array is disposed, said ridge extending circumferentially about said array contiguously so as to define a container containing said probe array, said ridge spacing said probe surface and said opposing surface so that said opposing surface has an average distance above said probe region from said probe surface less than 200 microns.

2. A bio-analytic reaction system as recited in claim 1 wherein said ridge defines a closed figure so that said container encloses said probe array.

3. A bio-analytic system as recited in claim 1 wherein said ridge defines an open figure with end points so as to define a fluid communication passage into and out of said chamber, said open figure in combination with a straight line segment connecting said endpoints defining a closed figure so that said container encloses said probe array.

4. A bio-analytic system as recited in claim 1 wherein said average distance is less than 100 microns.

5. A bio-analytic system as recited in claim 1 wherein said average distance is less than 50 microns.

6. A bio-analytic system as recited in claim 1 wherein said ridge is compliant and provides a barrier between said sample liquid and the exterior of said reaction cell.

7. A bio-analytic system as recited in claim 1 further comprising a centrifuge for applying ultragravity centrifugal forces to said reaction cell.

8. A bio-analytic system as recited in claim 7 further comprising replenishment means for inducing a replenishment motion in a sample liquid within said chamber while said ultragravity centrifugal forces are being applied to said reaction cell.

9. A bio-analytic system as recited in claim 8 wherein said replenishment means achieves said replenishment motion by rocking said reaction cell relative to said ultragravity centrifugal forces.

10. A bio-analytic method comprising:
providing a reaction cell holding sample liquid, said cell having a probe-bearing surface and defining a chamber bounded by said probe-bearing surface, said chamber having an average height less than 200 microns above said probe-bearing surface;
centrifuging said chamber so as to apply ultragravity centrifugal forces to said sample liquid; and
inducing a replenishment motion in said sample liquid during said centrifuging.

11. A bio-analytic method as recited in claim 10 wherein said replenishment motion involves laminar flow so that at most partial mixing is achieved.

12. A bio-analytic method as recited in claim 10 wherein said replenishment motion is induced by moving said cell relative to said ultragravity centrifugal force.

13. A bio-analytic method as recited in claim 10 wherein said average height is less than 100 microns.

14. A bio-analytic method as recited in claim 13 wherein said average height is less than 50 microns.

15. A bio-analytic method as recited in claim 10 further comprising a step of purging said reaction cell by pivoting it so that it points downward relative to said ultragravity centrifugal force.

16. A bio-analytic method as recited in claim 10 wherein said reaction cell is sealed by a compliant ridge against loss of sample fluid during centrifuging.

17. A method as recited in claim 10 wherein said reaction cell has a substantially circumferential ridge spacing said probe surface from an opposing surface and defining a barrier between said sample liquid an the exterior of said reaction cell.

18. A bio-analytic system comprising: 80 a on the Shore “A” scale.
an array of bioactive probes;
a reaction cell having a probe surface, an opposing surface, and
a compliant ridge, said probe surface having a probe region on which said array is disposed, said ridge spacing said probe surface and said opposing surface so that said opposing surface has an average distance above said probe region from said probe surface less than 200 microns.

19. A bio-analytic system as recited in claim 18 wherein said ridge encloses said probe array.

20. A bio-analytic system as recited in claim 18 wherein said ridge defines an open figure with end points so as to define a fluid communication passage into and out of said chamber, said open figure in combination with a straight line segment connecting said endpoints defining a closed figure that encloses said probe array.

21. A bio-analytic system as recited in claim 18 further comprising a centrifuge for applying ultragravity centrifugal forces to said reaction cell.

22. A bio-analytic system as recited in claim 21 further comprising replenishment means for inducing a replenishment motion in a sample liquid within said chamber while said ultragravity centrifugal forces are being applied to said reaction cell.

23. A bio-analytic system as recited in claim 21 wherein said mixing means achieves replenishment by rocking said reaction cell relative to said ultragravity centrifugal forces.
